(19) United States
(12) Patent Application Publication
(10) Pub. No.: US 2006/0079825 A1
(43) Pub. Date: Apr. 13, 2006

(54) Overlay garment for improving muscle tone

(76) Inventors: Christine Hilton, Rural Retreat, VA (US); Mildred L. McClure, Marion, VA (US)

Correspondence Address: WILLIAMS MULLEN, FOUNTAIN PLAZA THREE, SUITE 200, 721 LAKEFRONT COMMONS, NEWPORT NEWS, VA, 23606, US

(21) Appl. No.: 10/962,869

(22) Filed: Oct. 12, 2004

(51) Int. Cl. A61F 13/00; A61F 13/06

(52) U.S. Cl. 602/67; 602/60; 602/61

(57) ABSTRACT

A sensory pelvic garment for pediatric patients with various pressure panels to assist with inhibition or facilitation for hypotonic or hypertonic muscle tone. The garment is used to treat pediatric patients with neurological muscular impairments by providing pressure to inhibit abnormal muscle tone and to facilitate postural awareness.

### FIELD OF THE INVENTION

The invention is generally related to an overlay garment. More particularly, the invention is directed to an overlay garment for improving muscle tone in children with neurological muscular impairments.

### DESCRIPTION OF RELATED ART

A certain segment of the pediatric population suffers from sensory neurological impairments. While numerous devices exist for providing orthopedic support or treatment, none exist which provide appropriate treatment for neurological impairments, particularly for pediatric patients.

U.S. Pat. No. 5,188,585 describes an apparatus for providing orthopedic support for the lower back. The apparatus is specifically made for pain relief and does not provide neurological stimulation.

U.S. Pat. No. 4,696,291 describes another orthopedic support device for the lower back. Again, the device does not provide neurological stimulation but is meant to treat lower back pain.

U.S. Pat. No. 4,715,364 device which locks the pelvic bones into a desired orientation. This device can be used to retain the body in a desired configuration during movement or to prevent lumbago. No neurological stimulation is described.

U.S. Pat. No. 5,569,171 describes a chiropractic brace for orthopedic support of the back. The device is designed to support the spinal column and prevent injury due to movement and is rigid rather than flexible. This device does not provide neurological stimulation.

U.S. Pat. No. 5,950,628 describes a wearable traction device for treating maternal, back or wrist pain. The device is designed to prevent movement of the injured area during healing. The device does not provide neurological stimulation.

U.S. Patent Application No. 2001/0027149 describes a frame and body suit for exercising neurologically impaired persons. The body suit is suspended from the frame to provide gravitational support to the suspended person, allowing them to exercise their muscles when they are unable to support their full body weight. While the device allows the user to exercise and, hopefully, strengthen muscles, direct neurological stimulation is not provided.

Accordingly, it is an object of the present invention to provide a method and device for providing neurological stimulation to neurologically impaired pediatric patients.

It is another object of the present invention to provide a method and device which inhibit abnormal muscle tone.

It is another object of the present invention to provide a method and device which inhibit abnormal movement patterns.

It is another object of the present invention to provide a method and device for treating neurological impairments by means of pressure applied to selected muscles.

It is a further object of the present invention to provide a method and device for treating neurological impairments by providing sensory feedback with respect to selected pressure point.

Finally, it is an object of the present invention to accomplish the foregoing objectives in a safe, simple and cost effective manner.

### SUMMARY OF THE INVENTION

A method for improving muscle tone in neurological patients in which a band of flexible, stretchable material having a memory characteristic includes a closure means for connecting the opposing ends of the band. The band is placed around the pelvic girdle of a neurological patient and secured around the patient with the closure means. The band provides pressure against the patient, preferably along the entire length of the band. The band is preferably made from spandex material and the closure is preferably hook and loop fastener, snaps, buttons or hooks. This method can be used for treating neurological impairments in hypotonic patients.

In an alternate embodiment, the band consists of a first portion which surrounds the patient's pelvic girdle and a second portion which extends from the lower edge of the front of the pelvic girdle portion to the lower edge of the back of the pelvic girdle portion. The second portion includes a compression panel which extends substantially from the superior sacrum to the ischial tuberosities of the patient. If desired, the portions in combination can be designed in the form of a diaper overlay. This method can be used for treating neurological impairments in hypertonic patients.

### BRIEF DESCRIPTION OF THE DRAWINGS

FIG. 1 is front view of the preferred embodiment of the present invention;

FIG. 2 is a rear view of the preferred embodiment of the present invention;

FIG. 3 is a side view of the preferred embodiment of the present invention;

FIG. 4 is a front view of an alternate embodiment of the present invention;

FIG. 5 is a rear view of the alternate embodiment of the present invention shown in FIG. 4; and

FIG. 6 is a side view of the alternate embodiment of the present invention shown in FIG. 4.

### ELEMENT LIST

12 Band providing pressure to pelvic girdle
14 Closure means
16 Band providing pressure from superior sacrum to ischial tuberosities
18
20
22
24

### DETAILED DESCRIPTION OF THE PREFERRED EMBODIMENT

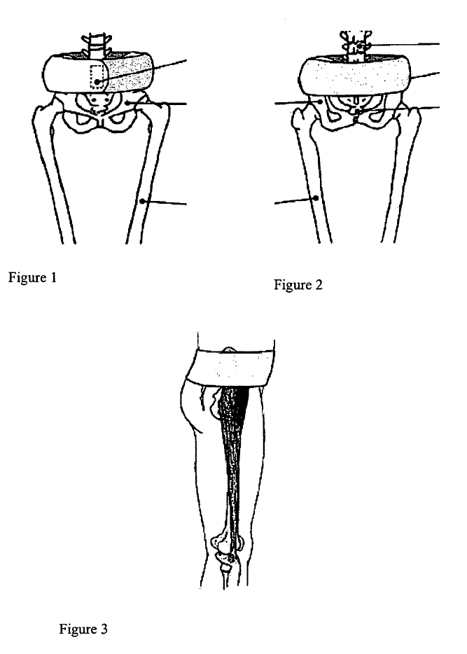

Referring now to the drawings, FIGS. 1-6 show two embodiments of the present invention. FIGS. 1-3 show (a preferred embodiment of the invention. This embodiment provides a consistent and constant pressure around the waist, lateral hips, sacral/coccyx region and the anterior pelvis and, in use, essentially encases the pelvic girdle. In use, the device provides pressure to facilitate tactile, vestibular and proprioceptive senses. In use, the device causes the wearer to be aware of posture. Because awareness is heightened, muscular tone is improved. This embodiment of the invention is particularly helpful in treating pediatric patients with hypotonic muscle tone.

As shown, the invention consists of a band 12 which wraps around the pelvic girdle of the patient. The band 12 is made from a stretchy, flexible material, such as spandex. The band 12 is designed to provide constant pressure along its diameter when in use. Thus, the material preferably stretches along its length but includes a memory characteristic encouraging return to its unstretched configuration in order to provide the necessary pressure.

The ends of the band are connected by a removable means 14. Hook and loop fastener is preferably used but other methods such as snaps, hooks or buttons may also be appropriate.

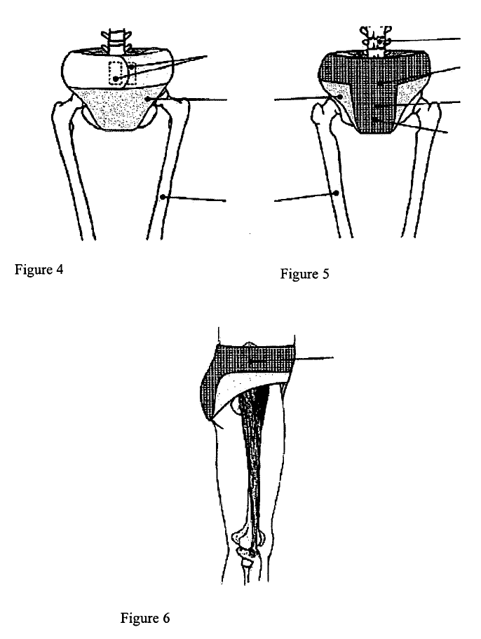

FIGS. 4-6 show an alternate embodiment of the present invention. This embodiment is designed for treating hypertonic and hemiplegic patients with neurological impairments. This embodiment encases the posterior pelvis and ends at the lateral aspect of the hips providing pressure to the lateral rotators and abductor musculature. The embodiment produces increased posterior and lateral pressure, forcing the patient into an anterior pelvic tilt and assists with the inhibition of adductor tone.

This embodiment is designed particularly for pediatric patients and is in the form of a diaper overlay. A band 12 of material, which is flexible and stretches as described previously, again surrounds the pelvic girdle of the patient. An additional portion of similar material 16 reaches from the superior sacrum to the ischial tuberosities.

Again, the ends of the band 12,16 are connected by a removable means 14. Hook and loop fastener is preferably used but other methods such as snaps, hooks or buttons may also be appropriate.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method for treating neurological impairments comprising the steps of:
providing a band of flexible, stretchable material having a memory characteristic;
providing a closure means for connecting a first end of the band to a second end of the band;
placing the band around the pelvic girdle of a neurological patient; and
enabling the closure means to secure the band around the patient such that the band provides pressure against the patient.

2. The method as set forth in claim 1, wherein the band is made from spandex material.

3. The method as set forth in claim 1 wherein the closure means is selected from the group consisting of hook and loop fastener, snaps, buttons and hooks.

4. The method as set forth in claim 1 wherein the band provides constant pressure against the patient along the length of the band.

5. The method as set forth in claim 1 wherein the band comprises:
a first length which surrounds the pelvic girdle of the patient; and
a second length which extends from the lower edge of the front of the pelvic girdle length to the lower edge of the back of the pelvic girdle length.

6. The method as set forth in claim 5 wherein the second length further comprises:
a compression panel which extends substantially from the superior sacrum to the ischial tuberosities of the patient.

7. The method as set forth in claim 5 wherein the band is designed as a diaper overlay.

8. A method for treating neurological impairments in hypotonic patients comprising the steps of:
providing a means for inducing pressure about the pelvic girdle of the patient; and
securing the pressure inducing means to the patient.

9. A method for treating neurological impairments in hypertonic patients comprising the steps of:
providing a first means for inducing pressure about the pelvic girdle of the patient;
providing a second means for inducing pressure from the superior sacrum to the ischial tuberosities of the patient; and
securing the first and second pressure inducing means to the patient.

10. A device for treating neurological impairments in patients, comprising:
a band of flexible, stretchable material having a memory characteristic; and
a closure means for connecting a first end of the band to a second end of the band such that the band encircles the pelvic girdle of a neurological patient and provides pressure against the patient.

11. The device as set forth in claim 10 wherein the band is made from spandex material.

12. The device as set forth in claim 10 wherein the closure means is selected from the group consisting of hook and loop fastener, snaps, buttons and hooks.

13. The device as set forth in claim 10 wherein the band provides constant pressure against the patient along the length of the band.

14. The device as set forth in claim 10 wherein the band comprises:
a first length which surrounds the pelvic girdle of the patient; and
a second length which extends from the lower edge of the front of the pelvic girdle length to the lower edge of the back of the pelvic girdle length.

15. The device as set forth in claim 14 wherein the second length further comprises:
a compression panel which extends substantially from the superior sacrum to the ischial tuberosities of the patient.

16. The device as set forth in claim 14 wherein the band is designed as a diaper overlay.
